(19) United States
(12) Patent Application Publication
(10) Pub. No.: US 2002/0197323 A1
(43) Pub. Date: Dec. 26, 2002

(54) Stable solid delivery system and method of preparing same

(76) Inventors: Subraman Rao Cherukuri, Vienna, VA (US); Stuart L. Cantor, Eldersburg, MD (US)

Correspondence Address: NATH & ASSOCIATES PLLC, 6th Floor, 1030 15th Street NW, Washington, DC, 20005, US

(21) Appl. No.: 09/886,473

(22) Filed: Jun. 22, 2001

(51) Int. Cl. A61K 9/14

(52) U.S. Cl. 424/488

(57) ABSTRACT

A novel process for preparing a stable solid delivery system in which the steps include: blending about 30.0 to 90.0% by weight a carbohydrate component with about 2.0 to 12.0% by weight a humectant component and remainder water in a heating vessel to form a mixture; heating the mixture to a final temperature of about 150° F. to about 300° F. to form a cooked mixture; cooling the cooked mixture while continually mixing to a temperature of about 175° F. to 250° F. to form a cooled mixture; blending about 0.5 to 30.0% by weight of an emulsifier system; maintaining a temperature of about 175° F. to 250° F. and blending said emulsifier system with the cooled mixture to form a delivery base; cooling the delivery base to a temperature below about 110° F. to form a stable solid delivery system; and mixing the stable solid delivery system with at least one active agent.

### BACKGROUND OF THE INVENTION

1. Field of the Invention

The present invention relates to a stable solid delivery system and a method of preparing same. In particular, the present invention relates to a stable solid delivery system prepared from high temperature emulsions.

2. Description of the Prior Art

Pharmaceutical compositions may be produced in a variety of dosage forms, depending upon the desired route of administration of the therapeutic material. Oral dosage forms, for example, include such solid compositions as tablets, emulsions, and suspensions. The particular dosage form utilized will depend on such factors as the solubility and chemical reactivity of the pharmaceutical active. Further, the dosage form may be selected so as to optimize delivery of the pharmaceutical active and/or consumer acceptability of the composition.

Tablet compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active. However, liquid formulations may offer advantages in the treatment of certain disorders, such as disorders of the upper gastrointestinal tract, wherein delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed.

A further product often used to deliver active agents to patients are emulsions. An emulsion is a dispersed system containing at least two immiscible liquids. The majority of conventional emulsions in pharmaceutical use have dispersed particles ranging in diameter from 0.1 to 100 microns. As with suspensions, emulsions are thermodynamically unstable as a result of the excess free energy associated with the surface of the particles. The dispersed particles, therefore, strive to come together and reduce the surface area. In addition to this flocculation effect, the dispersed particles can coalesce, or fuse, and this can result in the eventual destruction of the emulsion. In order to minimize this effect, a third component, the emulsifying agent, is added to the system to improve stability. The choice of emulsifying agent is critical to the preparation of an emulsion possessing optimum stability.

Invariably, one of the two emulsified components is aqueous, while the other component is a fatty substance, such as an oil. Many aqueous emulsions are prepared at elevated temperatures, that is at temperatures greater than 175° F. The elevated temperature aids in the dispersal of the non-aqueous component into the aqueous component by the emulsifying agent. However, if the high temperature emulsion is cooled in order to solidify the mixture, separation of the fatty substance, or “oiling off” may observed in the final product if the levels of the fatty substance are too high or if the fatty substance is not added at the proper temperature.

A further disadvantage of high temperature emulsions is the detrimental effect of the high temperature on the efficacy and stability of active agents added to the emulsion. Many active agents, whether the active agent is a flavor, pharmaceutical, or nutriceutical, are not stable at high temperatures. Thus, if the active agents are added at the high temperatures, the active agents break down, resulting in uneven dosing (or doses that contain no active agents) and waste of the active agents.

Furthermore, many pharmaceutical and nutriceutical active agents have a bitter taste, so a flavor-enhancer or taste-masking agent is also added to oral doses containing the pharmaceutical or nutriceutical, resulting in extra care needed to ensure that neither the pharmaceutical or nutriceutical, nor the flavor enhancer is destroyed during processing of the high temperature emulsion prior to cooling to a solid base.

Thus, there is a need for a stable solid delivery system prepared from a high temperature emulsion in which the fatty component of the emulsion does not separate or “oil off” when the emulsion is cooled to form the solid delivery system. Further, there is a need for a stable solid delivery system prepared from a high temperature emulsion in which the active agents are not deteriorated or destroyed during the high temperature emulsion process.

It has been found that the present inventive process for preparing a stable solid delivery system from a high temperature emulsion results in a product with no separation of the fatty component. Further, the active agents present in the stable solid delivery system are not deteriorated or destroyed by the high temperatures used in preparing the stable solid delivery system. Other objects and advantages of the present inventive subject matter are expressed herein.

### BRIEF SUMMARY OF THE INVENTION

Applicant has unexpectedly developed a process for preparing a stable solid delivery system comprising the steps of:

a) blending about 30.0 to 90.0% by weight a carbohydrate component with about 2.0 to 12.0% by weight a humectant component and remainder water in a heating vessel to form a mixture;

b) heating said mixture to a final temperature of about 150° F. to about 300° F. to form a cooked mixture;

c) cooling said cooked mixture while continually mixing to a temperature of about 175° F. to 250° F. to form a cooled mixture;

d) blending about 0.5 to 30.0% by weight of an emulsifier system;

e) maintaining a temperature of about 175° F. to 250° F. and blending said emulsifier system with said cooled mixture to form a delivery base;

f) cooling said delivery base to a temperature below about 110° F. to form a stable solid delivery system; and

g) mixing said stable solid delivery system with at least one active agent.

Applicant has further developed a process for preparing a stable solid delivery system comprising the steps of:

a) blending about 30.0 to 90.0% by weight a carbohydrate component with about 2.0 to 12.0% by weight a humectant component and remainder water in a heating vessel to form a mixture;

b) heating said mixture to a final temperature of about 150° F. to about 300° F. to form a cooked mixture;

c) cooling said cooked mixture while continually mixing to a temperature of about 175° F. to 250° F. to form a cooled mixture;

d) blending about 0.5 to 30.0% by weight of an emulsifier system;

e) maintaining a temperature of about 175° F. to 250° F. and blending said emulsifier system with said cooled mixture to form a delivery base;

f) cooling said delivery base to a temperature below about 110° F. to form a stable solid delivery system; and

g) forming said stable solid delivery system into a desired shape and cooling same to room temperature;

h) reheating said stable solid delivery system to a temperature of about 110° F.; and

i) mixing said stable solid delivery system with at least one active.

Further, Applicant has unexpectedly developed a process for preparing a pharmaceutical composition comprising the steps of:

a) blending about 30.0 to 90.0% by weight a carbohydrate component with about 2.0 to 12.0% by weight a humectant component and remainder water in a heating vessel to form a mixture;

b) heating said mixture to a final temperature of about 150° F. to about 300° F. to form a cooked mixture;

c) cooling said cooked mixture while continually mixing to a temperature of about 175° F. to 250° F. to form a cooled mixture;

d) blending about 0.5 to 30.0% by weight of an emulsifier system;

e) maintaining a temperature of about 175° F. to 250° F. and blending said emulsifier system with said cooled mixture to form a delivery base;

f) cooling said delivery base to a temperature below about 110° F. to form a stable solid delivery system; and

g) mixing said stable solid delivery system with at least one therapeutically active substance to form said pharmaceutical composition.

Still further, Applicant has unexpectedly developed a process for preparing a pharmaceutical composition comprising the steps of:

a) blending about 30.0 to 90.0% by weight a carbohydrate component with about 2.0 to 12.0% by weight a humectant component and remainder water in a heating vessel to form a mixture;

b) heating said mixture to a final temperature of about 150° F. to about 300° F. to form a cooked mixture;

c) cooling said cooked mixture while continually mixing to a temperature of about 175° F. to 250° F. to form a cooled mixture;

d) blending about 0.5 to 30.0% by weight of an emulsifier system;

e) maintaining a temperature of about 175° F. to 250° F. and blending said emulsifier system with said cooled mixture to form a delivery base;

f) cooling said delivery base to a temperature below about 110° F. to form a stable solid delivery system; and

g) forming said stable solid delivery system into a desired shape and cooling same to room temperature;

h) packaging said stable solid delivery system for transportation to a remote location;

i) transporting said stable solid delivery system to said remote location;

j) removing said stable solid delivery system from said packaging;

k) heating said stable solid delivery system to a temperature of about 110° F.; and

l) mixing said base with a therapeutically active substance to form said pharmaceutical composition.

### DETAILED DESCRIPTION OF THE EMBODIMENTS

The inventive process for preparing the stable solid delivery system includes blending a carbohydrate component with a humectant component and remainder water in a heating vessel to form a mixture, then heating the mixture to a final temperature of about 150° F. to about 300° F. to form a cooked mixture, cooling the cooked mixture while continually mixing to a temperature of about 175° F. to 250° F. to form a cooled mixture, blending about 0.5 to 30.0% by weight of an emulsifier system, maintaining a temperature of about 175° F. to 250° F. and blending the emulsifier system with the cooled mixture to form a delivery base, cooling the delivery base to a temperature below about 110° F. to form a stable solid delivery system, and mixing the stable solid delivery system with at least one active agent.

As used herein, the expression “mammal” includes without limitation any mammalian subject, such as mice, rats, guinea pigs, cats, dogs, human beings, cows, horses, sheep or other livestock.

The expression “solid” includes items which are solid at room temperature, and those items which may be considered semisolid or soft chew at room temperature. In general, the term “solid” is meant to cover those items that are not liquid or gas at room temperature.

The expression “emulsifier system” means a component that comprises emulsifiers, fats and mixtures thereof.

As used herein, the expression “active agent” includes, without limitation, therapeutically active substances, vitamins, minerals, antibiotics, and mixtures thereof.

The unique process of the present inventive subject matter allows for complete emulsification of a fatty component in an aqueous environment without separation of the fat from the aqueous component when the temperature of the emulsion is lowered. Further, the unique process allows for incorporation of active agents into stable solid delivery system with no decomposition or destruction of the active agents due to the high temperatures of the emulsion.

The incorporation of the active agents after the stable solid delivery system has been formed provides great flexibility for the uses of the present inventive process. For example, the active agents may be incorporated into the stable solid delivery base immediately after cooling the base to a temperature below about 110° F., thus resulting in a final product which may be formed and packaged in conventional manners for shipment and delivery to a customer. In this example, the active agents and stable solid delivery system are mixed in the same plant or facility in which the stable solid delivery system was prepared. Also, the preparation of the stable solid delivery system and the mixing of the active agent are done within a short period of time (within a couple of hours) of each other.

Another advantage of the stable solid delivery system is that the active agents may be incorporated thereinto at a later time. For example, the stable solid delivery system may be prepared by the inventive process and intermediately packaged for storage. Then at a later date (days, weeks or months after initial preparation of the delivery system), the stable solid delivery system is reheated and the active agents are incorporated into the stable solid delivery system, resulting in a final product that is formed and packaged by conventional means and delivered to the customer. It is contemplated within the scope of the present inventive process that this later incorporation of the active agents into the stable solid delivery system may take place at the same site as did the preparation of the stable solid delivery system, or it may take place at a remote location.

As used herein, “remote location” means a location that is separate from the location in which the stable solid delivery system is prepared according to the present inventive process. The remote location may be another building within the same complex as the facility in which the delivery system was prepared, or it may be at a location that is away from the delivery system production site. Of course, if the remote location is a site that is away from the production site, then the intermediately packaged stable solid delivery system must be transported to the remote location. The present inventive subject matter contemplates all modes of transporting the stable solid delivery system to the remote location, including without limitation, by truck or other automotive vehicle, airplane, train, or ship.

The initial step of the present inventive process comprises blending about 30.0 to 90.0% by weight a carbohydrate component with about 2.0 to 12.0% by weight a humectant component and remainder water in a heating vessel to form a mixture. The term “carbohydrate” as used herein refers to a class of compounds comprising mono- and di-saccharides. Generally, the carbohydrate component is selected from the group consisting of dextrose, polysaccharides, high-maltose corn syrup, corn syrup, sugar-free components, edible polymers and mixtures thereof. This includes liquid syrups and crystalline or solid sugars. Further, the carbohydrate component may be liquid fructose (content is 99% fructose by weight), high fructose corn syrup, invert sugar, maltose syrup and sugar alcohols such as sorbitol, and mixtures thereof.

As is stated above, the carbohydrate component may be an edible polymer. Edible polymers refers to that class of polymers useful in the food and confectionery art. Examples of edible polymers besides polysaccharides include, without limitation, polysaccharide derivatives, proteins and glycerin. Useful polysaccharide derivatives include, for example, starch, gelatine, carboxy methyl cellulose, ethyl methyl cellulose, cream of tartar, hydroxy propyl methyl cellulose, carrageenan gum, sodium alginate, xanthan gum, guar gum, gum arabic, and locus bean gum.

The second component mixed in the initial step of the inventive process is 2.0 to 12.0% by weight a humectant. The humectant may be in solid or liquid form. The humectant is selected from the group consisting of hydrogenated starch hydrolysate, maltitol, lactitol, glycerin, sorbitol, xylitol, mannitol and mixtures thereof. It has been found that the carbohydrates and humectants act synergistically together to produce a base which is flexible and non-staling over time. Also, the synergistic effect produces a base that does not stick to teeth or packaging materials.

The remainder of the initial mixture which is blended together in the first step of the inventive process is water. The water may be present in quantities up to about 10% by weight.

After the components have been blended together, the mixture is then heated to a final temperature of about 150° F. to about 300° F. Preferably, the mixture is heated to a temperature of about 230° F. to about 270° F. This forms a cooked mixture in which the heat has removed most of the water from the initial mixture.

The cooked mixture is next cooled to a temperature of about 175° F. to about 250° F., thus forming a cooled mixture. The cooling of the cooked mixture is done while the cooked mixture is still being stirred or mixed. This ensures proper blending and mixing of the components in the mixture.

The fourth step of the present inventive process comprises blending about 0.5 to 30.0% by weight of an emulsifier system. The emulsifier system comprises emulsifiers, fats and mixtures thereof. Generally, the emulsifier system comprises 0.5 to 10.0% (by weight of the final composition) of at least one emulsifier and 7.0 to 20.0% by weight (also of the final composition) of at least one fat.

The function of the emulsifier is to prevent the oil or fat phase from separating from the carbohydrate/water phase of the product. The emulsifier also provides good aeration in the buccal cavity during chewing. Emulsifiers work well to provide a smooth mouthfeel and help prevent the product from sticking to the packaging materials. However, there is a critical level of usage for the emulsifier beyond which the beneficial effect of the emulsifier will be negated. In other words, too high a percentage of emulsifiers will result in a reduction of the bite force of the stable solid delivery system and act to create a softer product.

Examples of emulsifiers that work well in the present inventive process include, without limitation, acetylated monoglycerides, glycerol esters, lecithin, de-oiled lecithin, enzyme-modified lecithins, purified lecithins, glyceryl monostearate, polyglycerol esters, propylene glycol esters, sorbitan esters, polysorbate esters, sodium laurel sulfate and mixtures thereof. Selection of the proper emulsifier will depend on the desired characteristics of the final stable solid delivery system.

The emulsifier system also includes at least one fat component. The fats are chosen based on their solid fat index (SFI), active oxygen stability and melting characteristics at mammalian body temperature. The fat component of the emulsifier system acts to improve the pliability of the final stable solid delivery system. However, if the level of fats in the delivery system is too high, or the fats are added at too high of a temperature, separation or “oiling off” is observed during handling of the stable solid delivery system. By adding the emulsifier system at the correct temperature, the oil-soluble compounds become embedded in the carbohydrate matrix and form an oil-in-water emulsion.

Examples of fats that may be used in the emulsifier system include, without limitation, chocolate, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate and mixtures thereof. Selection of the fat component for use in the emulsifier system will depend on the desired characteristics of the final stable solid delivery system.

After the emulsifier system has been blended, the temperature is maintained at about 175° F. to about 250° F. and the emulsifier system is blended with the cooled mixture to form a delivery base. The delivery bases is then cooled to a temperature below about 110° F to form a stable solid delivery system. The stable solid delivery system then may be formed into a desired shape.

The stable solid delivery system is next mixed with at least one active agent. As is explained above, the active agent may be mixed with the delivery system immediately after preparation of the delivery system, or at a later time.

In a preferred embodiment of the present inventive process, the stable solid delivery system is formed into a desired shape and cooled to room temperature. After a period of time, the stable solid delivery system is reheated to a temperature of about 110° F. and at least one active agent is mixed therewith to form the final product. The reheating of the stable solid delivery system may take place at a remote location, as is discussed above.

In another preferred embodiment of the present inventive process, the stable solid delivery system is formed into a desired shape and cooled to room temperature, after which the stable solid delivery system is packaged for transport to a remote location. After the stable solid delivery system is transported to the remote location, it is removed from the packaging and heated to a temperature of about 110° F. and mixed with at least one therapeutically active substance to form a pharmaceutical composition.

In all of the above embodiments of the present inventive subject matter, at least one active agent is mixed with the stable solid delivery system. Generally, the active agent is selected from the group consisting of therapeutically active substances, vitamins, minerals, antacids, cough and cold medications, analgesics, cardiovasular medications, anti-smoking, psycho-therapeutics, antibiotics, and mixtures thereof.

Examples of therapeutically active substances that may be active agents in the present inventive process include, without limitation, antitussives, antihistamines, decongestants, alkaloids, mineral supplements, laxatives, vitamins, antacids, ion exchange resins, anti-cholesterolemics, antiarrhythmics, antipyretics, analgesics including acetaminophen, aspirin, non-asteroidal anti-inflammatory drugs (“NSAID”) and opioids, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

Further preferred nutritional active materials useful in the present inventive subject matter include, without limitation, calcium-containing materials such as calcium carbonate, vitamins, minerals, herbals, spices and mixtures thereof.

Examples of vitamins that are available as active ingredients include, without limitation, vitamin A (retinol), vitamin D (cholecalciferol), vitamin E group (α-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

The present inventive process for preparing a stable solid delivery system also contemplates adding additional ingredients along with the active agent. The additional ingredients are selected from the group consisting of colors, flavors, sweeteners, surfactants, preservatives, bulking agents, and mixtures thereof

Flavors may be chosen from natural and synthetic flavor liquids. Flavors useful in the present inventive process include, without limitation, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof.

Further examples of flavors useful in the inventive process include, without limitation, beef flavorings, chicken flavorings, rice flavorings, lamb flavorings, pork flavorings, seafood flavorings, and mixtures thereof.

The sweeteners may be chosen from the following non-limiting list: flucose (corn syrup), dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol, and the like. Also contemplated are hydrogenated starch hydrolysates and synthetic sweetener 3,6-dihydro-6-methyl-1-1-1,2,3-oxathiazin-4-one-2,2-dioxide, particularly the potassium salt (acesulfame-K) and sodium and calcium salts thereof. Other sweeteners may also be used.

The present inventive subject matter also contemplates the stable solid delivery systems produced by the inventive process.

The following examples are illustrative of preferred embodiments of the invention and are not to be construed as limiting the invention thereto. All percentages are given in weight percent, unless otherwise noted and equal a total of 100%. In all Examples, Brix refers to a hydrometer scale for sugar solutions so graduated that its readings at a specified temperature represent percentages by weight of sugar in the solution.

### EXAMPLES

### Example #1

### Preparation of Stable Solid Delivery System #1

1352.00 pounds of corn syrup and 447.20 pounds of water were added to a heating vessel. Following mixing of the corn syrup and water, 696.8 pounds of granulated sugar were added, along with 104.00 pounds of sorbitol. The mixture was heated to about 240° F. and a Brix of 87 to form a cooked mixture.

The cooked mixture was cooled to about 185-230° F. 176.04 pounds of partially hydrogenated soybean oil, 29.34 pounds of glycerol mono-stearate, and 14.58 pounds of lecithin were mixed. The fats were mixed for two minutes until homogeneous. The fats were then mixed with 1580.04 pounds of the cooled cooked mixture. The mixture was allowed to cool, forming a stable solid delivery system.

The stable solid delivery system was molded and allowed to set-up.

### Example #2

### Preparation of Stable Solid Delivery System #2

1352.00 pounds of corn syrup and 447.20 pounds of water were added to a heating vessel. Following mixing of the corn syrup and water, 696.8 pounds of granulated sugar were added, along with 104.00 pounds of sorbitol. The mixture was heated to about 240° F. and a Brix of 87 to form a cooked mixture.

The cooked mixture was cooled to about 185-230° F. 19.41 pounds of partially hydrogenated soybean oil, 3.24 pounds of glycerol mono-stearate, 1.62 pounds of lecithin, and 32.25 pounds of chocolate liquor were mixed in a separate cooker. The fats were mixed for two minutes until homogeneous. The fats were then mixed with 174.31 pounds of the cooled cooked mixture.

The mixture was allowed to cool, forming a stable solid delivery system.

The stable solid delivery system was molded and allowed to set-up.

### Example #3

### Preparation of Stable Solid Delivery System #3

1352.00 pounds of corn syrup and 447.20 pounds of water were added to a heating vessel. Following mixing of the corn syrup and water, 696.8 pounds of granulated sugar were added, along with 104.00 pounds of sorbitol. The mixture was heated to about 240° F. and a Brix of 87 to form a cooked mixture.

The cooked mixture was cooled to about 185-230° F. 476.59 grams of partially hydrogenated soybean oil, and 53.61 grams of lecithin were mixed in a separate cooker. The fats were mixed for two minutes until homogeneous. The fats were then mixed with 4168.46 grams of the cooled cooked mixture. The mixture was allowed to cool, forming a stable solid delivery system.

The stable solid delivery system was molded and allowed to set-up.

### Example #4

### Preparation of Stable Solid Delivery System for Glucosamine

566.16 grams of the stable solid delivery system were prepared in accordance with Example #3 and added to a sigma mixer. To the mixer was added 73.26 grams of sugar, 3.20 grams of caramel flavor, 2.88 grams of vanilla flavor and 1.04 grams of vanillin. The mixture was blended until homogenous.

To the mixture was added 125.84 grams of encapsulated glucosamine, and 27.62 grams of flavors, sweeteners and colors. This mixture was blended until homogeneous and allowed to set up. The final product was formed and packaged in 5.5-6.0 gram-sized pieces.

### Example #5

### Preparation of Stable Solid Delivery System for Glucosamine

567.78 grams of the stable solid delivery system were prepared in accordance with Example #1 and added to a sigma mixer. To the mixer was added 65.30 grams of sugar, 1.28 grams of caramel flavor, 4.08 grams of vanilla flavor and 1.04 grams of vanillin. The mixture was blended until homogenous.

To the mixture was added 125.84 grams of encapsulated glucosamine, and 34.68 grams of flavors, sweeteners and colors. This mixture was blended until homogeneous and allowed to set up. The final product was formed and packaged in 5.5-6.0 gram-sized pieces.

### Example #6

### Preparation of Stable Solid Delivery System for Calcium and Vitamin D₃

578.77 pounds of the stable solid delivery system were prepared in accordance with Example #1 and added to a sigma mixer. To the mixer was added 8.00 pounds of sugar, 3.60 pounds of caramel flavor, and 0.96 pounds of vanillin. The mixture was blended until homogenous.

To the mixture was added 188.96 pounds of calcium carbonate, and 19.71 grams of flavors, sweeteners and colors. This mixture was blended until homogeneous and allowed to set up. The final product was formed into the desired shape.

### Example #7

### Preparation of Stable Solid Delivery System for Calcium and Vitamin D₃

385.74 pounds of the stable solid delivery system were prepared in accordance with Example #3 and added to a sigma mixer. To the mixture were added 5.60 pounds of sugar, 3.68 pounds of caramel flavor, and 0.96 pounds of vanillin

To the mixture was added 188.96 pounds of calcium carbonate, and 45.30 pounds of flavors, sweeteners and colors. This mixture was blended until homogeneous and allowed to set up. The final product was formed into the desired shape.

### Example #8

### Preparation of Stable Solid Delivery System for Zinc, Echinacea and Menthol

424.34 pounds of the stable solid delivery system were prepared in accordance with Example #3 and added to a sigma mixer. To the mixture were added 145.54 pounds of sugar, 3.60 pounds of caramel flavor, 4.00 pounds of vanilla flavor, and 0.96 pounds of vanillin

To the mixture was added 14.54 pounds of echinacea, 2.20 pounds of zinc acetate dihydrate, and 46.72 pounds of flavors, sweeteners and colors were added to the mixture. This mixture was blended until homogeneous and allowed to set up. The final product was formed into the desired shape.

The inventive subject matter being thus described, it will be obvious that the same may be varied in many ways. Such variations are not to be regarded as a departure from the spirit and scope of the inventive subject matter, and all such modifications are intended to be included within the scope of the following claims.

1. A process for preparing a stable solid delivery system comprising the steps of:
a) blending about 30.0 to 90.0% by weight a carbohydrate component with about 2.0 to 12.0% by weight a humectant component and remainder water in a heating vessel to form a mixture;
b) heating said mixture to a final temperature of about 150° F. to about 300° F. to form a cooked mixture;
c) cooling said cooked mixture while continually mixing to a temperature of about 175° F. to 240° F. to form a cooled mixture;
d) blending about 0.5 to 30.0% by weight of an emulsifier system;
e) maintaining a temperature of about 175° F. to 240° F. and blending said emulsifier system with said cooled mixture to form a delivery base;
f) cooling said delivery base to a temperature below about 110° F. to form a stable solid delivery system; and
g) mixing said stable solid delivery system with at least one active agent.

2. The process according to claim 1 wherein said emulsifier system contains at least one emulsifier and at least one fat.

3. The process according to claim 2 where in said emulsifier is present in an amount from about 0.5 to 20.0% by weight of the final composition and said fat is present in an amount from about 1.0 to 10.0% by weight of the final composition.

4. The process according to claim 1 wherein said final temperature in step b) is from about 230° F. to about 270° F.

5. The process according to claim 1, wherein said active agent is selected from the group consisting of therapeutically active substances, vitamins, minerals, antacids, cough and cold medications, analgesics, cardiovasular medications, antismoking, psycho-therapeutics, antibiotics, and mixtures thereof.

6. The process according to claim 1 wherein additional ingredients are added in step g), said additional ingredients selected from the group consisting of colors, flavors, sweeteners, surfactants, preservatives, bulking agents, and mixtures thereof.

7. The process according to claim 1, wherein said carbohydrate component is selected from the group consisting of dextrose, polysaccharides, high-maltose corn syrup, corn syrup, sugar-free components, edible polymers and mixtures thereof.

8. The process according to claim 1, wherein said humectant component is selected from the group consisting of hydrogenated starch hydrolysate, maltitol, lactitol, glycerin, sorbitol, xylitol, mannitol and mixtures thereof.

9. The process according to claim 2, wherein said fat component is selected from the group consisting of chocolate, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate and mixtures thereof.

10. The process according to claim 2, wherein said emulsifier is selected from the group consisting of acetylated monoglycerides, glycerol esters, lecithin, de-oiled lecithin, enzyme-modified lecithins, purified lecithins, glyceryl monostearate, polyglycerol esters, propylene glycol esters, sorbitan esters, polysorbate esters, sodium laurel sulfate and mixtures thereof.

11. The process according to claim 1, further comprising the step of forming said stable solid delivery system into a desired shape.

12. A process for preparing a stable solid delivery system comprising the steps of:
a) blending about 30.0 to 90.0% by weight a carbohydrate component with about 2.0 to 12.0% by weight a humectant component and remainder water in a heating vessel to form a mixture;
b) heating said mixture to a final temperature of about 150° F. to about 300° F. to form a cooked mixture;
c) cooling said cooked mixture while continually mixing to a temperature of about 175° F. to 250° F. to form a cooled mixture;
d) blending about 0.5 to 30.0% by weight of an emulsifier system;
e) maintaining a temperature of about 175° F. to 250° F. and blending said emulsifier system with said cooled mixture to form a delivery base;
f) cooling said delivery base to a temperature below about 110° F. to form a stable solid delivery system; and
g) forming said stable solid delivery system into a desired shape and cooling same to room temperature;
h) reheating said stable solid delivery system to a temperature of about 110° F.; and
i) mixing said stable solid delivery system with at least one active.

13. The process according to claim 12 wherein said emulsifier system contains at least one emulsifier and at least one fat.

14. The process according to claim 13 where in said emulsifier is present in an amount from about 0.5 to 20.0% by weight of the final composition and said fat is present in an amount from about 1.0 to 10.0% by weight of the final composition.

15. The process according to claim 12 wherein said final temperature in step b) is from about 230° F. to about 270° F.

16. The process according to claim 12, wherein said active agent is selected from the group consisting of therapeutically active substances, vitamins, minerals, antacids, cough and cold medications, analgesics, cardiovasular medications, antismoking, psycho-therapeutics, antibiotics, and mixtures thereof.

17. The process according to claim 12 wherein additional ingredients are added in step g), said additional ingredients selected from the group consisting of colors, flavors, sweeteners, surfactants, preservatives, bulking agents, and mixtures thereof.

18. The process according to claim 12, wherein said carbohydrate component is selected from the group consisting of dextrose, polysaccharides, high-maltose corn syrup, corn syrup, sugar-free components, edible polymers and mixtures thereof.

19. The process according to claim 12, wherein said humectant component is selected from the group consisting of hydrogenated starch hydrolysate, maltitol, lactitol, glycerin, sorbitol, xylitol, mannitol and mixtures thereof.

20. The process according to claim 13, wherein said fat component is selected from the group consisting of chocolate, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate and mixtures thereof.

21. The process according to claim 13, wherein said emulsifier is selected from the group consisting of acetylated monoglycerides, glycerol esters, lecithin, de-oiled lecithin, enzyme-modified lecithins, purified lecithins, glyceryl monostearate, polyglycerol esters, propylene glycol esters, sorbitan esters, polysorbate esters, sodium laurel sulfate and mixtures thereof.

22. The process according to claim 12, wherein said reheating occurs at a different physical location than the blending steps.

23. A process for preparing a pharmaceutical composition comprising the steps of:
a) blending about 30.0 to 90.0% by weight a carbohydrate component with about 2.0 to 12.0% by weight a humectant component and remainder water in a heating vessel to form a mixture;
b) heating said mixture to a final temperature of about 150° F. to about 300° F. to form a cooked mixture;
c) cooling said cooked mixture while continually mixing to a temperature of about 175° F. to 250° F. to form a cooled mixture;
d) blending about 0.5 to 30.0% by weight of an emulsifier system;
e) maintaining a temperature of about 175° F. to 250° F. and blending said emulsifier system with said cooled mixture to form a delivery base;
f) cooling said delivery base to a temperature below about 110° F. to form a stable solid delivery system; and
g) mixing said stable solid delivery system with at least one therapeutically active substance to form said pharmaceutical composition.

24. The process according to claim 23 wherein said emulsifier system contains at least one emulsifier and at least one fat.

25. The process according to claim 24 where in said emulsifier is present in an amount from about 0.5 to 20.0% by weight of the final composition and said fat is present in an amount from about 1.0 to 10.0% by weight of the final composition.

26. The process according to claim 23, wherein said active agent is selected from the group consisting of therapeutically active substances, vitamins, minerals, antacids, cough and cold medications, analgesics, cardiovasular medications, anti-smoking, psycho-therapeutics, antibiotics, and mixtures thereof.

27. The process according to claim 23 wherein additional ingredients are added in step g), said additional ingredients selected from the group consisting of colors, flavors, sweeteners, surfactants, preservatives, bulking agents, and mixtures thereof.

28. The process according to claim 23, wherein said carbohydrate component is selected from the group consisting of dextrose, polysaccharides, high-maltose corn syrup, corn syrup, sugar-free components, edible polymers and mixtures thereof.

29. The process according to claim 23, wherein said humectant component is selected from the group consisting of hydrogenated starch hydrolysate, maltitol, lactitol, glycerin, sorbitol, xylitol, mannitol and mixtures thereof.

30. The process according to claim 24, wherein said fat component is selected from the group consisting of chocolate, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate and mixtures thereof.

31. The process according to claim 23, wherein said emulsifier is selected from the group consisting of acetylated monoglycerides, glycerol esters, lecithin, de-oiled lecithin, enzyme-modified lecithins, purified lecithins, glyceryl monostearate, polyglycerol esters, propylene glycol esters, sorbitan esters, polysorbate esters, sodium laurel sulfate and mixtures thereof.

32. A process for preparing a pharmaceutical composition comprising the steps of:
a) blending about 30.0 to 90.0% by weight a carbohydrate component with about 2.0 to 12.0% by weight a humectant component and remainder water in a heating vessel to form a mixture;
b) heating said mixture to a final temperature of about 150° F. to about 300° F. to form a cooked mixture;
c) cooling said cooked mixture while continually mixing to a temperature of about 175° F. to 250° F. to form a cooled mixture;
d) blending about 0.5 to 30.0% by weight of an emulsifier system;
e) maintaining a temperature of about 175° F. to 250° F. and blending said emulsifier system with said cooled mixture to form a delivery base;
f) cooling said delivery base to a temperature below about 110° F. to form a stable solid delivery system; and
g) forming said stable solid delivery system into a desired shape and cooling same to room temperature;
h) packaging said stable solid delivery system for transportation to a remote location;
i) transporting said stable solid delivery system to said remote location;
j) removing said stable solid delivery system from said packaging;
k) heating said stable solid delivery system to a temperature of about 110° F.; and
l) mixing said base with a therapeutically active substance to form said pharmaceutical composition.

33. The composition produced by the process of claim 1.

34. The composition produced by the process of claim 12.

35. The composition produced by the process of claim 23.

36. The pharmaceutical composition produced by the process of claim 32.

37. The pharmaceutical composition produced by the process of claim 28.
